(19) United States
(12) Patent Application Publication
(10) Pub. No.: US 2012/0107386 A1
(43) Pub. Date: May 3, 2012

(54) COMPOSITION AND METHOD FOR INCORPORATING NUTRIENTS INTO CONDIMENTS AND USE THEREOF IN COOKED FOODS

(75) Inventor: Edward Bukstel, Norristown, PA (US)

(73) Assignee: VITAMINSPICE LLC, Norristown, PA (US)

(21) Appl. No.: 13/320,714

(22) PCT Filed: May 14, 2010
(86) PCT No.: PCT/US10/34938

§ 371 Date: Jan. 12, 2012

(30) Foreign Application Priority Data
May 15, 2009 (US) 61178725

(51) Int. Cl. A23L 1/30; A23L 1/22; A61K 9/00

(52) U.S. Cl. 424/439; 426/96; 426/99; 426/103; 426/589

(57) ABSTRACT

A composition comprising a nutrient encapsulated in a coating and a condiment is disclosed. A method of manufacturing the composition is also disclosed.

### CROSS-REFERENCE TO RELATED APPLICATION

This application claims priority from U.S. Provisional Application Ser. No. 61/178,725, filed May 15, 2009, which is incorporated by reference herein in its entirety.

### BACKGROUND OF THE INVENTION

Many people, including children and adults, do not receive enough vitamin and mineral nutrition in their diets. One method for providing nutrition into the body is via capsules and pills that have specific amounts of dosage specifications of potentially numerous vitamins and minerals. One shortcoming of existing nutrient delivery methods is that they do not allow for individual people to vary the dosage of their vitamins. The existing systems utilize set dosages of vitamins that are contained in the capsules or pills. In the case of nutrients, which are incorporated into food products, the dosages of the incorporated nutrients are predetermined in portion sizes and ready to eat meals. In addition, many nutrients cannot withstand the temperatures of cooked foods.

It would be beneficial to provide a food additive in which a user can vary the amount of nutrient added to food based on specific needs or desires.

### BRIEF SUMMARY OF THE INVENTION

Briefly, the present invention provides a composition comprising a nutrient encapsulated in a coating and a condiment.

Further, the present invention provides a method of manufacturing a composition comprising the steps of encapsulating a nutrient in a coating; and mixing the coated encapsulated nutrient with a condiment.

Additionally, the present invention provides a composition manufactured according to the method described above.

### BRIEF DESCRIPTION OF THE DRAWINGS

The foregoing summary, as well as the following detailed description of the invention, will be better understood when read in conjunction with the appended drawings. For the purpose of illustrating the invention, there are shown in the drawings certain embodiments of the present invention. It should be understood, however, that the invention is not limited to the precise arrangements and instrumentalities shown. In the drawings, the same reference numerals are employed for designating the same elements throughout the several figures. In the drawings:

FIG. 1 is a sectional view of a coated nutrient according to an exemplary embodiment of the present invention; and

FIG. 2 is a flowchart illustrating an exemplary method of manufacturing the coated nutrient of FIG. 1.

### DETAILED DESCRIPTION OF THE PREFERRED EMBODIMENT(S)

In describing the embodiments of the invention illustrated in the drawings, specific terminology will be used for the sake of clarity. However, the invention is not intended to be limited to the specific terms so selected, it being understood that each specific term includes all technical equivalents operating in similar manner to accomplish similar purpose. It is understood that the drawings are not drawn exactly to scale.

The following describes particular embodiments of the present invention. It should be understood, however, that the invention is not limited to the embodiments detailed herein. The present invention comprises enhancing a condiment, which is frequently added to food prior to eating and, in some cases, prior to cooking, with one or more additional nutrients, such as one or more vitamins, minerals, anti-oxidants, or even medicine or other therapeutic substances (hereinafter “nutrient”). Examples of condiments include, spices, such as garlic, pepper, red pepper, cumin, cinnamon, ginger, and paprika, salad dressings, butter, ketchup, olive oil, and mustard. The nutrient is mixed with the condiment to form a composition of the nutrient and the condiment.

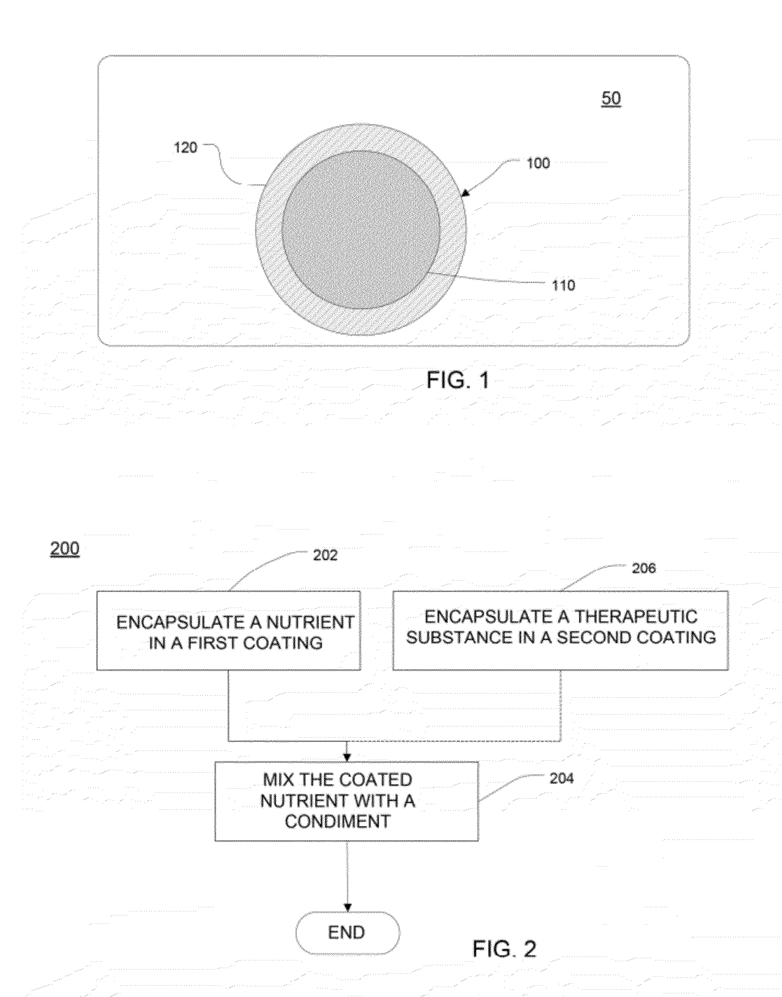

In one embodiment of a condiment 50 with a coated nutrient 100 according to an exemplary embodiment of the present invention, illustrated in FIG. 1, a nutrient 110 is provided in a powder or granular form and microencapsulated with a coating 120 that is intended to (1) enable the nutrient 110 to withstand cooking temperatures that the condiment 50 is likely to encounter during cooking, and (2) to mask the flavor of the nutrient 110. It is preferable for the coating 120 to enable the nutrient 110 to withstand temperatures of about 120 degrees F. without significant loss of nutritive value. A “significant loss of nutritive value” is defined herein as over 70% of a specific nutrient's nutritive value.

There are many microencapsulation methods know in the art that could be used to microencapsulate the nutrient. Spray-type microencapsulation method has been found to work well in several embodiments of the present invention. By way of example only, spray chilling and spray cooling with a fluid bed coating is a preferred encapsulation method for water soluble vitamins such as Vitamin C and B Vitamins. Utilization of a complex coacervation process or spray drying technology are preferred technologies for fat soluble vitamins such as Vitamin A, Vitamin D, Vitamin K.

Similarly, there are many potentially suitable coatings that could be applied to the nutrient powder. In addition to being suitable for microencapsulation applications, it is preferable that the coating be non-toxic, water and fat soluble, taste-neutral, provide acceptable taste masking properties, and desirable heat-protective properties. Optionally, the coating may have a taste that matches or complements the condiment with which the coated nutrient is to be mixed or combined. For example, if the coated nutrient is to be used with ketchup, the coating may have a taste similar to an ingredient in the ketchup.

Since the coating is intended to be ingested, it is also preferable that the coating be hypo-allergenic. Soybean oil and cellulose have been determined to provide good results, with cellulose being preferred because soy oil is an allergen to a higher percentage of potential consumers. Carboxymethylcellulose or protein and polysaccharide combinations may alternatively be used as coatings.

The desired thickness of the coating 120 will depend, in large part, on the harshness of the flavor of the nutrient 110 being coated and the temperature at which the bio-availability of the nutrient begins to degrade. All other variables being equal, nutrients having a harsher or more intense flavor will require a thicker coating than nutrients that are more flavor-neutral. Similarly, all other variables being equal, nutrients that begin to lose bio-availability at lower temperatures will require a thicker coating than nutrients that can withstand higher temperatures. Based on tests conducted on several common vitamins and minerals Vitamin C, Vitamin B12, Vitamin B6, niacin, iron, and magnesium, applying the coating at a thickness that corresponds to about 10-50%, by weight, of the coated nutrient powder (i.e., that the weight of the applied coating is 10-50% of the total weight of the coated nutrient) has provided acceptable results. For example, approximately 0.236 mg of a nutrient 110 could be coated with an equal weight amount of coating 120.

Once the nutrient has been microencapsulated, it can then be combined or mixed with the condiment, resulting in a “nutrient enriched condiment.” The preferred nutrient-to-condiment ratio is preferably selected to provide a desired amount of the nutrient on a daily basis, based on the expected usage of the condiment. Due to the volume and density differentials between condiments such as, for example, crushed red pepper versus garlic or cinnamon, the preferred ratio may differ. For instance, 0.236 milligrams (mg) of a vitamin 110 with a 0.236 mg coating 120 may be added to 4.2 grams of garlic. In another instance, 0.236 mg of a vitamin 110 with an equal amount of coating 120 may be added to 2.1 grams of crushed red pepper.

Several features of the inventive microencapsulated nutrient are that they may be kosher with all-natural spices, free of dairy and nuts, 100% vegetarian, free of additives, preservatives and coloring, low calorie or calorie-free, free of sugar and artificial sweeteners, and gluten-free.

The enriched condiment can then be applied to food based upon the individual user's taste preference. Accordingly, the enriched condiment allows a person to determine the dosage of the nutrient that is added to cooked food based upon their personal taste preferences.

In other embodiments, other therapeutic or medicinal substances, such as medicinal herbs or pharmaceuticals including, but not limited to gingko, rhodiola, St Johns Wort, saw palmetto, quercetin, glucosamine, methylsulfonylmethane, melatonin, ginseng, spirulina, phytochemicals, phytosterols, pre and probiotics, polyphenols, black cohosh and trace minerals, could be incorporated into the condiments instead of or in addition to nutrients. Accordingly, the invention could provide an alternative means of delivering therapeutic or medicinal substances, which could be beneficial in situations in which dosage compliance is a problem and/or if swallowing a liquid or pill-based medications is problematic. Such substances can be coated in accordance with the coatings and coating methods described above.

An exemplary method of providing a coated nutrient 100 is provided in flowchart 200 illustrated in FIG. 2. In step 202, a selected nutrient 110 is encapsulated in a coating 120 according to a process, such as the processes described above. In step 204, the coated nutrient 100 is mixed with a condiment 50 in a desired ratio, such as, for example, according to the type of condiment. Optionally, in step 206, a therapeutic substance, such as one of the therapeutic substances described above, may be coated with a second coating and mixed with the condiment.

In other embodiments, a condiment could be combined with a coated nutrient that provides benefits that are complementary to the condiment. The present invention enables such complementary combinations, even if the nutrient may not be taste-compatible with the condiment and/or could not withstand cooking temperatures without being coated. Examples of such combinations include:

TABLE 1

| Nutrient(s) | Condiment | Health benefit |
|---|---|---|
| chromium, magnesium, biotin, | cinnamon | healthy glucose |
| vitamin C, alpha lipoic acid, | | metabolism |
| and/or zinc | | |
| DHA or resveratrol | turmeric | maintaining good |
| | | memory |
| Coenzyme Q 10, Magnesium chelate, | garlic | hypertension |
| calcium citrate, taurine, Vitamin | | |
| D, L-arginine, and L-carnitine, | | |
| vitamin C, Vitamin E | | |
| caprylic acid, | garlic | treatment/prevention |
| | | of yeast infections |
| chromium, magnesium, biotin | cinnamon | hypertension |
| MSM (methylsulfonylmethane) | turmeric | muscle pain |

While the principles of the invention have been described above in connection with preferred embodiments, it is to be clearly understood that this description is made only by way of example and not as a limitation of the scope of the invention.

What is claimed is:

1. A composition comprising:
a nutrient encapsulated in a first coating; and
a condiment.

2. The composition according to claim 1, wherein the first coating masks the taste of the nutrient.

3. The composition according to claim 1, wherein the first coating weighs between about 10% and about 50% of the coated nutrient.

4. The composition according to claim 1, wherein the first coating enables the nutrient to withstand temperatures of about 120 degrees F. without losing over 70% of a nutritive value of the nutrient.

5. The composition according to claim 1, wherein the first coating is non-toxic, water and fat soluble, and taste-neutral.

6. The composition according to claim 1, wherein the first coating comprises soybean oil.

7. The composition according to claim 1, wherein the first coating comprises cellulose.

8. The composition according to claim 1, wherein the first coating comprises carboxymethylcellulose.

9. The composition according to claim 1, wherein the coating comprises protein and polysaccharide combinations.

10. The composition according to claim 1, further comprising a therapeutic substance encapsulated in a second coating.

11. A method of manufacturing a composition comprising the steps of:
a) encapsulating a nutrient in a first coating; and
b) mixing the coated encapsulated nutrient with a condiment.

12. The method according to claim 11, wherein the encapsulating step comprises masking the taste of the nutrient.

13. The method according to claim 11, wherein the encapsulating step comprises providing coating weighing between about 10% and about 50% of the coated nutrient.

14. The method according to claim 11, further comprising the steps of encapsulating a therapeutic substance in a second coating and mixing the coated encapsulated therapeutic substance with the condiment.

15. A composition manufactured by the process of:
a) encapsulating a nutrient in a coating; and
b) mixing the coated encapsulated nutrient with a condiment.
